(19) United States
(12) Patent Application Publication
(10) Pub. No.: US 2015/0191521 A1
(43) Pub. Date: Jul. 9, 2015

(54) DOMINANT NEGATIVE TRANSCRIPTION FACTOR FOR ANTAGONIZING ONCOGENIC TRANSCRIPTION FACTORS VIA MULTIMERIZATION

(71) Applicant: Cedars-Sinal Medical Center, Los Angeles, CA (US)

(72) Inventors: Norihiko Kawamata, Beverly Hills, CA (US); Phillip H. Koeffler, Los Angeles, CA (US)

(73) Assignee: Cedars-Sinal Medical Center, Los Angeles, CA (US)

(21) Appl. No.: 14/420,865

(22) PCT Filed: Aug. 27, 2013
(86) PCT No.: PCT/US13/56872

§ 371 Date: Feb. 10, 2015

Related U.S. Application Data
(60) Provisional application No. 61/693,640, filed on Aug. 27, 2012.

(51) Int. Cl. C07K 14/47; C07K 16/32; C07K 14/00

(52) U.S. Cl. CPC C07K 14/4746; C07K 14/4703; C07K 14/00; C07K 16/32; C07K 2319/80

(57) ABSTRACT

The present invention teaches compositions, methods and kits for treating or preventing cancer in a subject by using a dominant negative transcription factor and/or a multimerization adaptor. The invention teaches that the dominant negative transcription factor can antagonize an oncogenic transcription activator and can cause cell cycle arrest and cell death in a cancer cell. The invention further teaches delivering the inventive compositions by a variety of mechanisms, including: transfection, a virus-based system, and using a protein-transduction-domain moiety fused to the inventive dominant negative transcription factor.

### STATEMENT REGARDING FEDERALLY-SPONSORED RESEARCH

The U.S. Government has a paid-up license in this invention and the right in limited circumstances to require the patent owner to license others on reasonable terms as provided for by the terms of Grant No. 5R01CA026038-32 awarded by the National Cancer Institute.

### FIELD OF INVENTION

This invention generally relates to the treatment and prevention of cancer.

### BACKGROUND

Cancer cells can have a dysregulation of genes that can be caused by oncogenic transcription activators. There is a need in the art for therapies aimed at suppressing the function of these oncogenic transcription activators, and thereby suppressing proliferation of the cancer cells and/or causing cell-death.

### SUMMARY OF THE INVENTION

In various embodiments, the invention teaches a composition including a dominant negative transcription factor configured to antagonize an oncogenic transcription factor in a cancer cell or precancerous cell via multimerization. In some embodiments, the dominant negative transcription factor includes a multimerization domain and a DNA binding domain. In certain embodiments, the multimerization domain includes a tetramerization domain of p53. In various embodiments, the DNA binding domain includes an ETS-type binding domain. In some embodiments, the multimerization in a cancer cell or precancerous cell results in cell cycle arrest and/or cell death.

In various embodiments, the invention teaches a composition that includes a multimerization adaptor including one or more small molecules configured to multimerize an oncogenic transcription factor in a cancer cell. In some embodiments, one or more of the small molecules is selected from the group consisting of: adaptor proteins, antibodies, modified antibody-like molecules, and chemicals.

In various embodiments, the invention teaches a composition including a virus for facilitating the expression of one or more components of a dominant negative transcription factor and/or a multimerization adaptor, wherein the dominant negative transcription factor and/or multimerization adaptor are configured to antagonize an oncogenic transcription activator by inducing multimerization when expressed in a subject. In certain embodiments, the virus is selected from the group consisting of: a lentivirus, an adenovirus, an adeno-associated virus and a retrovirus.

In various embodiments, the invention teaches a method for treating or preventing cancer in a subject, including administering to the subject a composition including a means for expressing one or more component of a dominant negative transcription factor and/or a multimerization adaptor. In certain embodiments, the dominant negative transcription factor includes a multimerization domain and a DNA binding domain. In various embodiments, the multimerization domain includes a tetramerization domain of p53. In certain embodiments, the DNA binding domain includes an ETS-type DNA binding domain. In certain embodiments, the means for expressing the one or more component of the dominant negative transcription factor and/or the multimerization adaptor includes a virus. In certain embodiments, the virus is selected from the group consisting of: a lentivirus, an adenovirus, an adeno-associated virus and a retrovirus.

In various embodiments, the invention teaches a method for treating or preventing cancer in a subject, including administering to the subject a composition including a multimerization adaptor that includes one or more small molecules configured to multimerize an oncogenic transcription factor. In some embodiments, one or more of the one or more small molecules is selected from the group consisting of: adaptor proteins, antibodies, modified antibody-like molecules, and chemicals. In certain embodiments, the multimerization of the oncogenic transcription factor in a cancer cell results in cell cycle arrest and/or cell death.

In various embodiments, the invention teaches a kit for treating or preventing cancer in a subject. In some embodiments, the kit includes a composition including a means for expressing one or more components of a dominant negative transcription factor and/or a multimerization adaptor in the subject; and instructions for the use of the composition to treat and/or prevent cancer in the subject. In certain embodiments, the dominant negative transcription factor includes a multimerization domain and a DNA binding domain. In certain embodiments, the multimerization domain includes a tetramerization domain of p53. In various embodiments, the DNA binding domain includes an ETS-type DNA binding domain. In some embodiments, the means for expressing one or more components of the dominant negative transcription factor includes a virus.

In various embodiments, the invention teaches a kit for treating or preventing cancer in a subject. In some embodiments, the kit includes a composition that includes a multimerization adaptor that includes one or more small molecules configured to multimerize an oncogenic transcription factor in a cancer cell; and instructions for the use of the multimerization adaptor to treat or prevent cancer in the subject. In some embodiments, one or more of the small molecules is selected from the group consisting of: adaptor proteins, antibodies, modified antibody-like molecules, and chemicals. In certain embodiments, multimerization of the oncogenic transcription factor in a cancer cell results in cell cycle arrest and/or cell death.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 depicts, in accordance with an embodiment of the invention, dysregulation of genes caused by oncogenic transcription activators.

FIG. 2 depicts, in accordance with an embodiment of the invention, the difference in gene regulation between normal blood cells and leukemia cells and the effect of multimerization on downstream target genes.

FIG. 3 depicts, in accordance with an embodiment of the invention, an artificial dominant negative form of an oncogenic transcription factor made by fusing a well-characterized multimerization domain of p53 to an ETS-type DNA binding domain.

FIG. 4 depicts, in accordance with an embodiment of the invention, multimerization of an oncogenic transcription activator abolishes its transcription function.

FIG. 5 depicts, in accordance with an embodiment of the invention, inactivation of an oncogenic transcription activator facilitated by a multimerization adaptor.

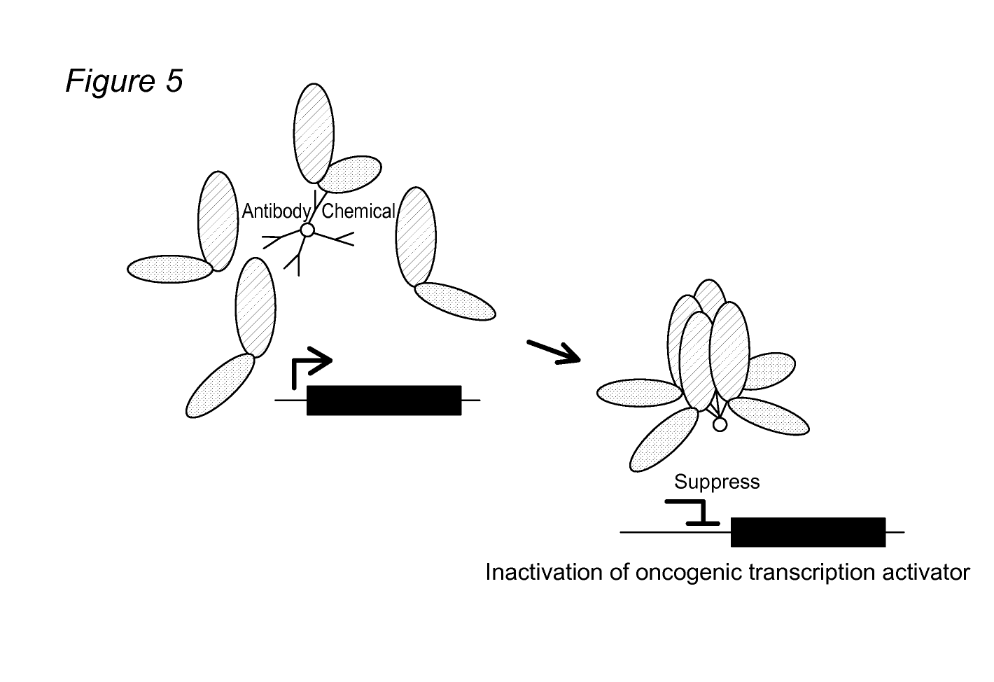

FIG. 6 depicts, in accordance with an embodiment of the invention, a strategy for introducing a dominant negative transcription factor into a cell.

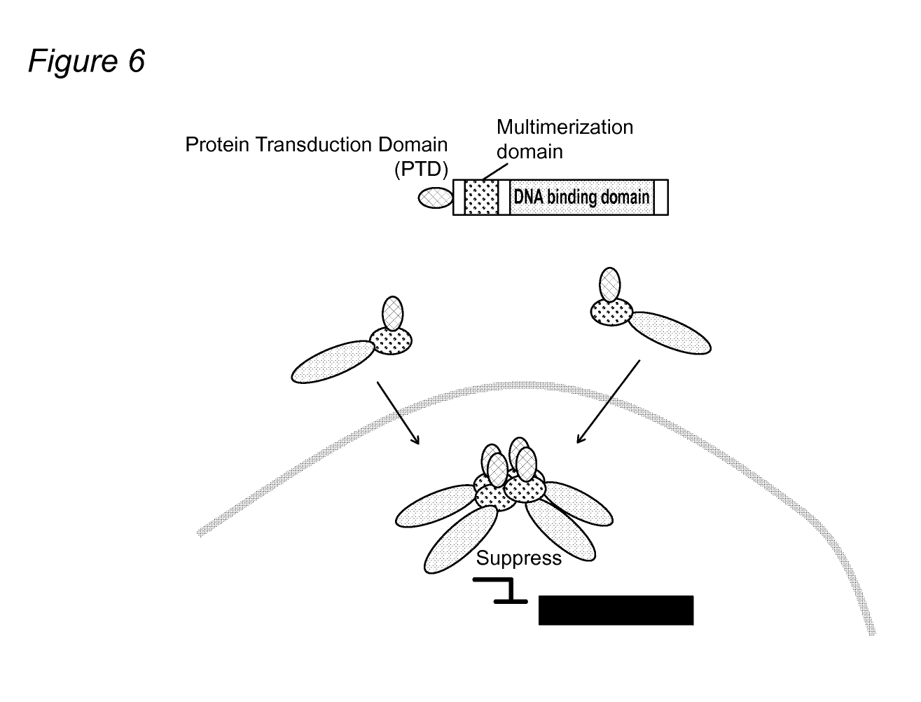

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russell, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, certain terms are defined below.

“Mammal” as used herein refers to a member of the class Mammalia, including, without limitation, humans, as well as nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, newborn subjects and infant subjects, as well as fetuses, whether male or female, are intended to be included within the scope of this term.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

By way of background, many oncogenic transcription activators function by strongly upregulating downstream target genes associated with cellular proliferation. This kind of regulation is associated with a number of different types of cancer well known to those of skill in the art.

In order to target this cause of cancer, the inventors designed an “artificial dominant negative form” of an oncogenic transcription factor by fusing a multimerization domain to a DNA binding domain from an oncogenic transcription factor. As demonstrated herein, multimerization can be accomplished in a variety of different ways, by using a number of different components that, when properly assembled, exert a dominant negative effect and thereby provide significant therapeutic value.

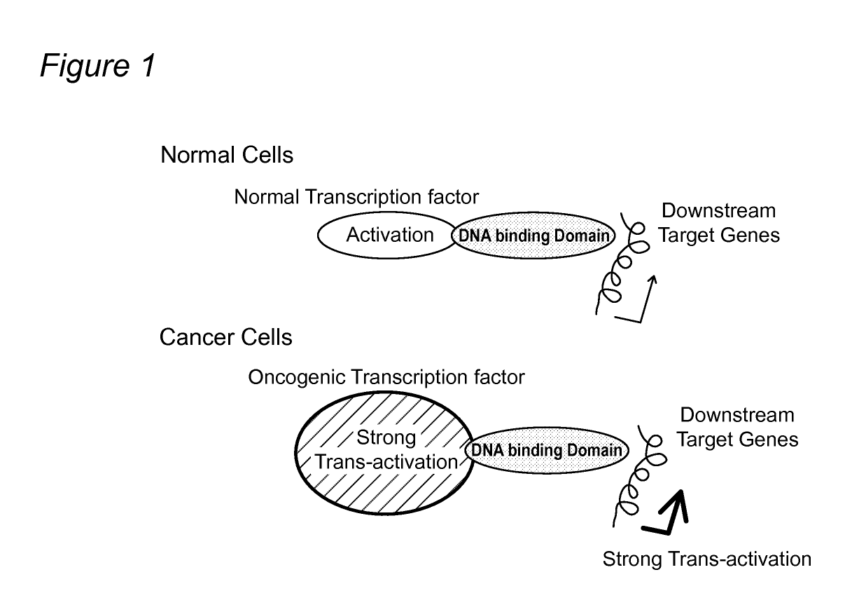

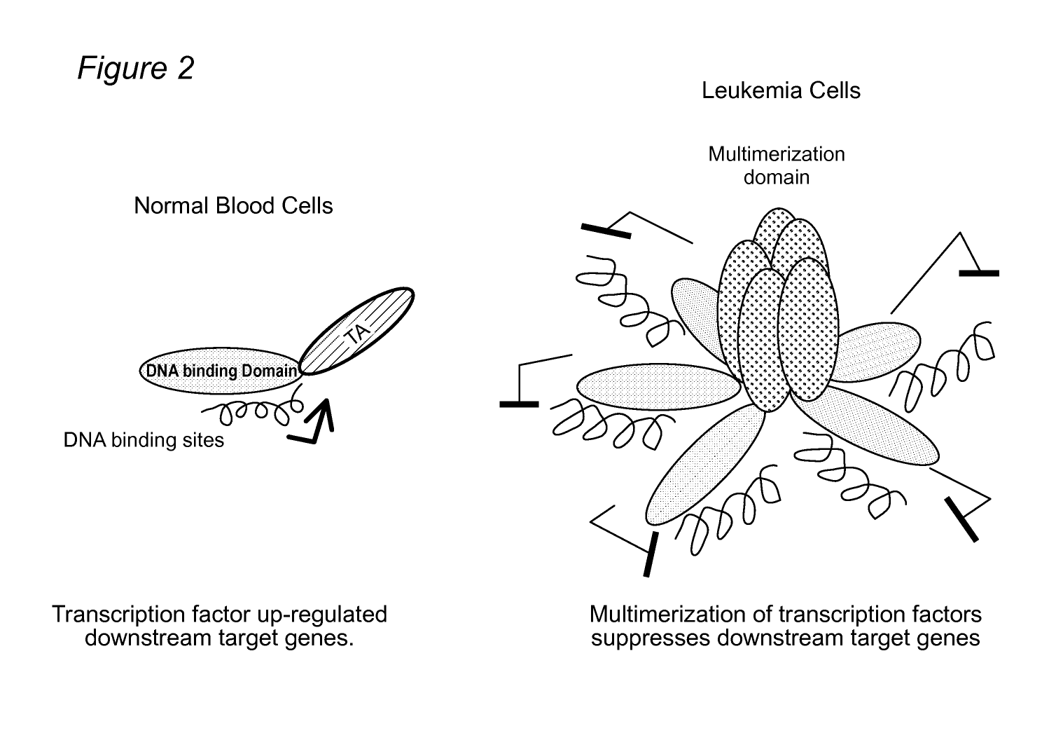

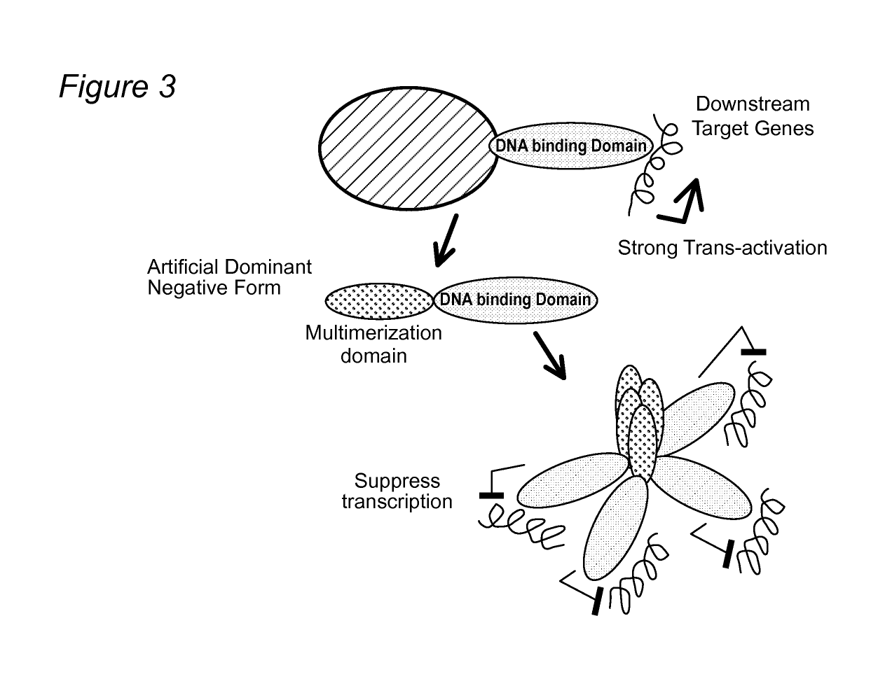

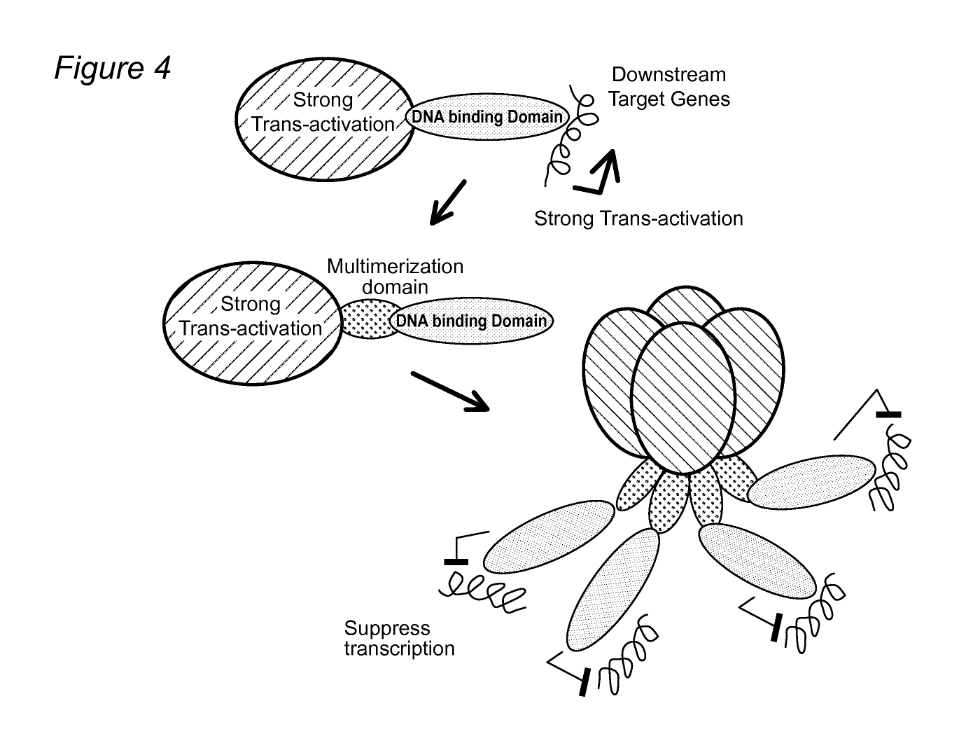

In some embodiments, the invention teaches a composition that includes a dominant negative transcription factor configured to antagonize an oncogenic transcription factor in a cancer cell or precancerous cell via multimerization, as demonstrated in FIGS. 1-4. In some embodiments, multimerization is characterized as the aggregation of from 2 to 6 molecules. One of skill in the art would readily appreciate that the exact number of molecules aggregated could vary greatly depending upon the components used. In certain embodiments, the dominant negative transcription factor includes a multimerization domain and a DNA binding domain. In some embodiments, the oncogenic transcription factor that is antagonized can include but is in no way limited to any of c-Myc, Fli-1, and the like.

In some embodiments, the multimerization domain is a tetramerization domain of p53. One of skill in the art would readily appreciate that alternative multimerization domains, whether natural or artificial, could be used to facilitate multimerization. Merely by way of example, the multimerization domain from C20orf112 protein, or the multimerization domain from LacZ protein could be used. One of skill in the art would readily appreciate that compounds capable of causing multimerization of transcription factors could be used as an alternative means of multimerization. In addition, a multimerized ligand can be generated, in which each ligand binds to each receptor-type transcription factor over-expressed in cancers. Merely by way of non-limiting example, the estrogen receptor is a nuclear-receptor type transcription factor that binds to estrogen. Estrogen binds to one molecule of estrogen receptor and works as a transcription factor as a monomer. Multimerized estrogen can be made, which can assemble multiple estrogen receptors, leading to strong DNA binding and transcriptional repression. These multimerized estrogen receptors can exert a dominant negative effect over the individual wild-type estrogen receptor.

In some embodiments, the DNA binding domain is an ETS-type DNA binding domain. One of skill in the art would readily appreciate that any of a number of DNA binding domains could be used to create dominant negative transcription factors for alternative targets using standard cloning techniques. Merely by way of example, c-Myc protein can be used. C-Myc is a strong transcription activator, and by inserting a multimerization domain into a c-Myc protein, it is possible to generate the dominant-negative form of c-Myc, which can inhibit the function of wild-type c-Myc over-expressed in a number of cancers. Yet another example is the estrogen receptor, a nuclear-receptor type transcription factor which is over-expressed in breast cancers. By inserting a multimerization domain, it is possible to generate the dominant-negative form of an estrogen receptor by multimerization, as described herein.

In various embodiments, when multimerization occurs in a cancer cell as a result of using the inventive methods and compositions, cell cycle arrest and/or cell death results.

In some embodiments, the invention teaches a multimer, including multiple subunits, wherein each of the subunits include a DNA binding domain and a multimerization domain, as described above.

In various embodiments, the invention teaches a multimerization adaptor, including one or more small molecules configured to multimerize an oncogenic transcription factor. In some embodiments, one or more of the small molecules can include but is in no way limited to any of the following: adaptor proteins, antibodies, modified antibody-like molecules, and chemicals. In some embodiments, a small peptide with both the DNA binding domain of Fli-1 and a multimerization domain is used. This small peptide antagonizes the effect of oncogenic transcription factor, Fli-1.

In various embodiments, the invention teaches a composition for expressing a dominant negative transcription factor and/or a multimerization adaptor in a subject. In some embodiments, the composition includes one or more vectors configured to express one or more components (i.e. subunits) of the dominant negative transcription factor and/or multimerization adaptor. In some embodiments, one or more of the vectors is a virus. In some embodiments the virus can include but is in no way limited to any of the following: a lentivirus, an adenovirus, an adeno-associated virus and a retrovirus. One of skill in the art would readily appreciate that alternative viruses could be used to facilitate the expression of the dominant negative transcription factor and/or the multimerization adaptor in a cell, and especially a cancer cell or a precancerous cell.

In various embodiments, the composition used to facilitate the expression of the dominant negative transcription factor and/or the multimerization adaptor may be provided as part of a pharmaceutical composition, including a pharmaceutically acceptable excipient. “Pharmaceutically acceptable excipient” means an excipient that is useful in preparing a composition that is generally safe, non-toxic, and desirable, and includes excipients that are acceptable for veterinary use as well as human use. Such excipients may be solid, liquid, semisolid, or, in the case of an aerosol composition, gaseous.

In various embodiments, the compositions described herein may be configured for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the composition may be in the form of solutions or suspensions for infusion or for injection, or as lyophilized powders. Via the enteral route, the composition can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the composition may be in the form of solutions or suspensions for infusion or for injection. Via the topical route, the compositions may be formulated for treating the skin and mucous membranes and is in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the indication.

The compositions according to the invention can also contain any pharmaceutically acceptable carrier. “Pharmaceutically acceptable carrier,” as used herein, refers to a pharmaceutically acceptable material, composition or vehicle that is involved in carrying or transporting the vector or multimerization adaptor from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its benefits to the animals described herein.

The compositions according to the invention can also be encapsulated, tableted or prepared in an emulsion or syrup for oral administration. Pharmaceutically acceptable solid or liquid carriers may be added to enhance or stabilize the compositions, or to facilitate preparation of the compositions described herein. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glycerylmonostearate or glyceryldistearate, alone or with a wax.

The preparations described herein are made following the conventional techniques of genetic manipulation and cloning, as well as those of pharmacy involving milling, mixing, granulation, and compressing, when necessary, for tablet forms; or milling, mixing and filling for hard gelatin capsule forms. When a liquid carrier is used, the preparation will be in the form of syrup, elixir, emulsion or an aqueous or non-aqueous suspension. Such a liquid formulation may be administered directly p.o. or filled into a soft gelatin capsule.

The compositions described herein may be delivered in an effective amount. The precise effective amount is that amount that will yield the most effective results in terms of efficacy of treatment in a given human or animal subject. This amount will vary depending upon a variety of factors, including but not limited to the characteristics of the composition (including activity and bioavailability), the physiological condition of the subject, the nature of the pharmaceutically acceptable carrier or carriers of the composition, and the route of administration.

In certain embodiments, the invention teaches a method for treating or preventing cancer in a subject, including administering a composition that facilitates the expression of a dominant negative transcription factor and/or a multimerization adaptor to the subject using one or more routes of administration described herein. In some embodiments, the dominant negative transcription factor includes a multimerization domain and a DNA binding domain. In some embodiments, the multimerization domain is the tetramerization domain of p53. Merely by way of non-limiting examples, the multimerization domain of C20orf112 protein, the multimerization domain of LacZ protein, or the like could also be used to facilitate multimerization. One of skill in the art would readily appreciate that alternative multimerization domains could also be used to facilitate multimerization without departing from the spirit of the invention. In some embodiments, the DNA binding domain is an ETS-type DNA binding domain. Merely by way of non-limiting examples, the c-Myc DNA binding domain, TCF4 DNA binding domain, and the like, can also be used in the inventive systems and methods described herein. One of skill in the art would readily appreciate that alternative DNA binding domains could also be used to create dominant negative transcription factors for alternative targets. In certain embodiments, the expression of the dominant negative transcription factor and/or the multimerization adaptor is accomplished using a virus. In some embodiments the virus can include but is in no way limited to any of the following: a lentivirus, an adenovirus, an adeno-associated virus and a retrovirus. One of skill in the art would readily appreciate that alternative viruses could be used to facilitate the expression of the dominant negative transcription factor and/or the multimerization adaptor in a cell, and especially a cancer cell or a precancerous cell. In some embodiments, the subject is a mammal. In some embodiments, the subject is a human.

In some embodiments, the invention teaches a method for treating or preventing cancer in a subject, including administering to the subject a composition including a protein transduction domain, a DNA binding domain and a multimerization domain. In some embodiments, the DNA binding domain and the multimerization domain are selected from those listed above and described herein. In certain embodiments, the protein transduction domain is a small peptide of approximately 8-12 amino acids that contains many arginine amino acids. One of skill in the art would readily appreciate that a number of alternative protein transduction domains of different sizes and compositions could be designed and used in conjunction with the invention. In some embodiments, the subject is a mammal. In some embodiments, the subject is a human.

In various embodiments, the invention teaches a method for treating or preventing cancer in a subject, including administering to the subject a composition that includes a multimerization adaptor, which includes one or more small molecules configured to multimerize an oncogenic transcription factor in a cancer cell. In some embodiments, one or more of the small molecules can include, but is in no way limited to any of the following: adaptor proteins, antibodies, modified antibody-like molecules, and chemicals. In some embodiments, the invention teaches a peptide which has a protein transduction domain (PTD), a multimerization domain, and the DNA binding domain of Fli1. This peptide can penetrate into the nucleus and inhibit the effect of the oncogenic transcription factor, Fli1. One of skill in the art would readily appreciate that similar peptides could be produced (using a PTD and multimerization domain) with different DNA binding domains, in order to inhibit the effect of different oncogenic transcription factors. In some embodiments, multimerization of the oncogenic transcription factor in a cancer cell results in cell cycle arrest and/or cell death. In some embodiments, the subject is a mammal. In some embodiments, the subject is a human.

In some embodiments, the molecules described herein are introduced into cancer cells or precancerous cells using transfection. In some embodiments, the molecules described herein are introduced into cancer cells or precancerous cells using a virus-based system (such as those indicated above). In some embodiments, the molecules described herein are introduced into cancer cells or precancerous cells using a protein-transduction-domain moiety fused to these molecules (such as those indicated above).

In various embodiments, the invention teaches a kit for treating and/or preventing cancer in a subject. In some embodiments, the kit includes a composition that facilitates the expression of a dominant negative transcription factor and/or a multimerization adaptor in the subject. In some embodiments, the dominant negative transcription factor includes a multimerization domain and a DNA binding domain. In some embodiments, the multimerization domain is a tetramerization domain of p53. One of skill in the art would readily appreciate that similar multimerization domains, such as any of those listed above, could be used to accomplish a similar result. In some embodiments, the DNA binding domain is an ETS-type DNA binding domain. One of skill in the art would readily appreciate that alternative DNA binding domains, such as any of those listed above, could be used to create dominant negative transcription factors for alternative targets. In certain embodiments, the expression of the dominant negative transcription factor and/or the multimerization adaptor is accomplished using a vector. In some embodiments, the vector is a virus. In some embodiments the virus can include, but is in no way limited to, any of the following: a lentivirus, an adenovirus, an adeno-associated virus and a retrovirus. One of skill in the art would readily appreciate that alternative viruses could be used to facilitate the expression of the dominant negative transcription factor in a cancer cell or precancerous cell. In some embodiments, the kit includes one or more of the multimerizing adaptors, including those described herein.

The exact nature of the components configured in the inventive kit depends on its intended purpose. For example, some embodiments are configured for the purpose of treating or preventing cancer. In some embodiments, the kit is configured for veterinary applications, treating subjects such as, but not limited to, farm animals, domestic animals, and laboratory animals. In some embodiments, the kit is configured for the purpose of treating human subjects.

Instructions for use may be included in the kit. “Instructions for use” typically include a tangible expression describing the technique to be employed in using the components of the kit to effect a desired outcome, such as to treat or prevent cancer. Instructions for use may include instructions to administer a single dose of any of the inventive compositions. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to a medical practitioner or other qualified individual stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in human medicine or veterinary medicine. As used herein, the term “package” refers to a suitable solid matrix or material such as glass, plastic, paper, foil, and the like, capable of holding the individual kit components. Thus, for example, a package can be one or more glass vials or plastic containers used to contain suitable quantities of the inventive composition. The packaging material generally has an external label which indicates the contents and/or purpose of the kit and/or its components.

One skilled in the art will recognize many methods and materials similar or equivalent to those described herein, which could be used in the practice of the present invention. Indeed, the present invention is in no way limited to the methods and materials described.

The following examples are for illustrative purposes only and are not intended to limit the scope of the disclosure or its various embodiments in any way.

### EXAMPLES

### Example 1

### Experiments

The inventors' previous research showed that leukemia cells often have chimeric transcription factors, composed of a DNA binding domain of important transcription factors and a multimerization domain from a partner protein. The inventors found that the chimeric transcription factors had a dominant negative effect over the wild-type transcription factors via multimerization. And, in this case, the wild-type transcription factors could not activate downstream target genes.

The inventors assumed that multimerization of the DNA binding domain from an oncogenic transcription activator could work as a dominant negative form over the oncoprotein, leading to cell death in cancer cells. To prove this idea, the inventors generated a dominant negative form of the Ews-Fli oncogenic transcription activator. Ews-Fli is the oncogenic transcription activator frequently detected in Ewing Sarcoma, among the most common type of bone cancers. The Ews region encodes a strong activation domain and the Fli region encodes an ETS-type DNA binding domain. This oncogenic transcription activator strongly upregulates downstream target genes associated with cellular proliferation. The inventors designed an “artificial dominant negative form” of this oncogenic transcription factor by fusing a well-characterized multimerization domain from p53 to the ETS-type DNA binding domain from this oncogenic transcription factor.

The inventors assumed that multimerization of an oncogenic transcription activator would abolish its transcription function. Consequently, the inventors generated a multimerization form of Ews-Fli, and confirmed it was inactive in terms of transcriptional activity.

In order to amplify the p53 multimerization domain, the following primers were used: 1) TP53 multimerization-S SEQ ID NO: 1—CCAGGGAGCACTAAGCGA and 2) TP53 multimerization-AS SEQ ID NO: 2—TCACCCTGGCTCCTTCCCAGC. To amplify the ETS domain in Fli1, the following primers were used: 1) ETS-S: SEQ ID NO: 3—AAGTCCTCCCCTTGGA and 2) ETS-AS: SEQ ID NO: 4—TTCTAGTAGTAGCTGCC.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A composition comprising a dominant negative transcription factor configured to antagonize an oncogenic transcription factor in a cancer cell or precancerous cell via multimerization.

2. The composition of claim 1, wherein the dominant negative transcription factor comprises a multimerization domain and a DNA binding domain.

3. The composition of claim 2, wherein the multimerization domain comprises a tetramerization domain of p53.

4. The composition of claim 2, wherein the DNA binding domain comprises an ETS-type binding domain.

5. The composition of claim 1, wherein the multimerization in a cancer cell or precancerous cell results in cell cycle arrest and/or cell death.

6. A composition comprising a multimerization adaptor comprising one or more small molecules configured to multimerize an oncogenic transcription factor in a cancer cell.

7. The composition of claim 6, wherein one or more of the small molecules is selected from the group consisting of: adaptor proteins, antibodies, modified antibody-like molecules, and chemicals.

8. A composition comprising a virus for facilitating the expression of one or more components of a dominant negative transcription factor and/or a multimerization adaptor, wherein the dominant negative transcription factor and/or multimerization adaptor are configured to antagonize an oncogenic transcription activator by inducing multimerization when expressed in a subject.

9. The composition of claim 8, wherein the virus is selected from the group consisting of: a lentivirus, an adenovirus, an adeno-associated virus and a retrovirus.

10. A method for treating or preventing cancer in a subject, comprising administering to the subject a composition comprising a means for expressing one or more component of a dominant negative transcription factor and/or a multimerization adaptor.

11. The method of claim 10, wherein the dominant negative transcription factor comprises a multimerization domain and a DNA binding domain.

12. The method of claim 11, wherein the multimerization domain comprises a tetramerization domain of p53.

13. The method of claim 11, wherein the DNA binding domain comprises an ETS-type DNA binding domain.

14. The method of claim 10, wherein the means for expressing the one or more component of the dominant negative transcription factor and/or the multimerization adaptor comprises a virus.

15. The method of claim 14, wherein the virus is selected from the group consisting of: a lentivirus, an adenovirus, an adeno-associated virus and a retrovirus.

16. A method for treating or preventing cancer in a subject, comprising administering to the subject a composition comprising a multimerization adaptor comprising one or more small molecules configured to multimerize an oncogenic transcription factor.

17. The method of claim 16, wherein one or more of the one or more small molecules is selected from the group consisting of: adaptor proteins, antibodies, modified antibody-like molecules, and chemicals.

18. The method of claim 16, wherein multimerization of the oncogenic transcription factor in a cancer cell results in cell cycle arrest and/or cell death.

19. A kit for treating or preventing cancer in a subject, comprising:
a composition comprising a means for expressing one or more components of a dominant negative transcription factor and/or a multimerization adaptor in the subject; and
instructions for the use of the composition to treat and/or prevent cancer in the subject.

20. The kit of claim 19, wherein the dominant negative transcription factor comprises a multimerization domain and a DNA binding domain.

21. The kit of claim 20, wherein the multimerization domain comprises a tetramerization domain of p53.

22. The kit of claim 20, wherein the DNA binding domain comprises an ETS-type DNA binding domain.

23. The kit of claim 19, wherein the means for expressing one or more components of the dominant negative transcription factor comprises a virus.

24. A kit for treating or preventing cancer in a subject, comprising:
a composition comprising a multimerization adaptor comprising one or more small molecules configured to multimerize an oncogenic transcription factor in a cancer cell; and
instructions for the use of the multimerization adaptor to treat or prevent cancer in the subject.

25. The kit of claim 24, wherein one or more of the small molecules is selected from the group consisting of: adaptor proteins, antibodies, modified antibody-like molecules, and chemicals.

26. The kit of claim 24, wherein multimerization of the oncogenic transcription factor in a cancer cell results in cell cycle arrest and/or cell death.


SEQUENCE LISTING (4 sequences)

SEQ ID NO: 1 - DNA, 18 bp, Homo sapiens
ccagggagcactaagcga

SEQ ID NO: 2 - DNA, 21 bp, Homo sapiens
tcaccctggctccttcccagc

SEQ ID NO: 3 - DNA, 16 bp, Homo sapiens
aagtcctccccttgga

SEQ ID NO: 4 - DNA, 17 bp, Homo sapiens
ttctagtagtagctgcc